(19) United States
(12) Patent Application Publication
(10) Pub. No.: US 2025/0380928 A1
(43) Pub. Date: Dec. 18, 2025

(54) ULTRASOUND PROBE

(71) Applicant: FUJIFILM Corporation, Tokyo (JP)

(72) Inventors: Gen SHIINA, Tokyo (JP); Takayuki IWASHITA, Tokyo (JP); Kenta YAMAGUCHI, Tokyo (JP); Takayoshi YANO, Tokyo (JP); Tsuneo KAWAMATA, Tokyo (JP)

(73) Assignee: FUJIFILM Corporation, Tokyo (JP)

(21) Appl. No.: 19/210,926

(22) Filed: May 16, 2025

(30) Foreign Application Priority Data
Jun. 17, 2024 (JP) 2024-097370

(51) Int. Cl. A61B 8/00

(52) U.S. Cl. CPC A61B 8/4455

(57) ABSTRACT

The ultrasound probe has an enlarged portion, a narrowed portion, and a body portion. A wide side surface of the enlarged portion consists of a front region and a rear region. The front region is flat. The rear region includes a step structure, a rear end region, one region, and the other region. The step structure includes a protrusion portion and a recess portion, in other words, a first step, a second step, and a third step. The height of each of the steps is 2.0 mm or less in a thickness direction.

### CROSS REFERENCE TO RELATED APPLICATION

This application claims priority to Japanese Patent Application No. 2024-097370 filed on Jun. 17, 2024, which is incorporated herein by reference in their entireties including the specifications, claims, drawings, and abstracts.

### BACKGROUND OF THE INVENTION

### 1. Field of the Invention

The present disclosure relates to an ultrasound probe, and more particularly to a form of an ultrasound probe.

### 2. Description of the Related Art

An ultrasound diagnostic apparatus is used for ultrasound examination of a living body. The ultrasound diagnostic apparatus is composed of an ultrasound diagnostic apparatus main body and an ultrasound probe. In general, an ultrasound probe is held by an examiner and is in contact with the surface of a living body. In this state, ultrasonic waves are transmitted from the ultrasound probe into the living body, and the reflected waves from within the living body are received by the ultrasound probe.

Ultrasound probes having a variety of forms are in use. A typical ultrasound probe is composed of a distal end portion and a grip portion. The distal end portion is a portion in which a transducer array is accommodated.

As a method of holding an ultrasound probe, there are various holding methods. Any holding method is selected depending on a posture of a subject, an examination site, a cross section to be observed, an examiner's preference, and the like. Recently, many examiners have employed a holding method that does not touch the surface of the subject.

Due to the background such as an increase in the amount of electronic components accommodated within an ultrasound probe and a request to move the center of gravity of the ultrasound probe closer to the distal end surface of the ultrasound probe in order to improve a decrease in the operability caused by the probe cable (specifically, instability of the ultrasound probe), there is a tendency for the distal end portion of the ultrasound probe to become enlarged. In addition, Design Registration No. 1329228, Design Registration No. 1470968, Design Registration No. 1314934, Design Registration No. 1387378, Design Registration No. 1457814, and Design Registration No. 1697765 disclose ultrasound probes.

### SUMMARY OF THE INVENTION

In a case in which the distal end portion of an ultrasound probe is held with a plurality of fingers, it is conceivable to provide an uneven structure on each side surface of the distal end portion so that the distal end portion can be held firmly. However, in a case in which the uneven structure has a large height difference (change in probe thickness), the large height difference may restrict the method of holding the ultrasound probe, or may cause the examiner to feel uncomfortable during the process of operating of the ultrasound probe. Furthermore, in a case in which the uneven structure has a large height difference, the work of removing an acoustic jelly that has adhered to the uneven structure becomes troublesome. On the other hand, in a case in which each side surface of the distal end portion is made completely flat (that is, in a case in which there is no unevenness on each side surface), a plurality of fingertips holding the ultrasound probe will tend to slip over the surface of the ultrasound probe in a case of pressing the ultrasound probe against the body surface or a case of removing the ultrasound probe from the body surface.

An object of the present disclosure is to provide an ultrasound probe that can appropriately prevent fingertip slippage while allowing a variety of holding methods. Alternatively, an object of the present disclosure is to provide an ultrasound probe having an anti-slip structure in a local region where slippage should be prevented within a region where a fingertip comes into contact.

According to an aspect of the present disclosure, there is provided an ultrasound probe having a central axis direction, a width direction, and a thickness direction that are orthogonal to each other, the ultrasound probe comprising: an enlarged portion that accommodates a transducer array; a narrowed portion that is continuous with the enlarged portion in the central axis direction; and a body portion that is continuous with the narrowed portion in the central axis direction, in which the enlarged portion has a distal end surface that intersects with respect to the central axis direction and includes an ultrasonic wave transmissive region, and two side surfaces that bulge out to both sides in the thickness direction, each of which intersects with respect to the thickness direction and extends along the central axis direction and the width direction, at least one side surface of the two side surfaces has a front region that is continuous with the distal end surface and is flat, and a rear region that is continuous with the front region in the central axis direction and has a step structure, the step structure has a protrusion portion that is provided on a rear side of the front region in the central axis direction, extends along the width direction, and has a band shape, a recess portion that is provided on a rear side of the protrusion portion in the central axis direction, extends along the width direction, allows a fingertip of an operator to be inserted, and has a band shape, and a rear end region that is provided on a rear side of the recess portion in the central axis direction and is flat, and a height of a first step between the front region and the protrusion portion, a height of a second step between the protrusion portion and the recess portion, and a height of a third step between the recess portion and the rear end region are all 2.0 mm or less in the thickness direction.

According to the aspect of the present disclosure, it is possible to provide an ultrasound probe that can appropriately prevent fingertip slippage while allowing a variety of holding methods. Alternatively, according to the aspect of the present disclosure, it is possible to provide an ultrasound probe having an anti-slip structure in a local region where slippage should be prevented within a region where a fingertip comes into contact.

### BRIEF DESCRIPTION OF THE DRAWINGS

FIG. 1 is a perspective view of an ultrasound probe according to an embodiment.

FIG. 2 is a diagram showing a state in which the ultrasound probe according to the embodiment is used.

FIG. 3 is a front view of the ultrasound probe according to the embodiment.

FIG. 4 is a side view of the ultrasound probe according to the embodiment.

FIG. 5 is a plan view of the ultrasound probe according to the embodiment.

FIG. 6 is a diagram showing a side surface of an enlarged portion.

FIG. 7 is a cross-sectional view of a step structure.

FIG. 8 is a diagram showing a substrate group accommodated across an enlarged portion and a narrowed portion.

### DESCRIPTION OF THE PREFERRED EMBODIMENTS

Hereinafter, an embodiment will be described with reference to the drawings.

### (1) Overview of Embodiment

An ultrasound probe according to an embodiment has a central axis direction, a width direction, and a thickness direction that are orthogonal to each other, and includes an enlarged portion that accommodates a transducer array, a narrowed portion that is continuous with the enlarged portion in the central axis direction, and a body portion that is continuous with the narrowed portion in the central axis direction. The enlarged portion has a distal end surface that intersects with respect to the central axis direction and includes an ultrasonic wave transmissive region, and two side surfaces that bulge out to both sides in the thickness direction, each of which intersects with respect to the thickness direction and extends along the central axis direction and the width direction. At least one side surface of the two side surfaces has a flat front region that is continuous with the distal end surface, and a rear region that is continuous with the front region in the central axis direction and has a step structure. The step structure has a band-shaped protrusion portion that is provided on the rear side of the front region in the central axis direction and extends along the width direction, a band-shaped recess portion that is provided on the rear side of the protrusion portion in the central axis direction, extends along the width direction, and allows a fingertip of an operator to be inserted, and a flat rear end region that is provided on the rear side of the recess portion in the central axis direction. A height of a first step between the front region and the protrusion portion, a height of a second step between the protrusion portion and the recess portion, and a height of a third step between the recess portion and the rear end region are all 2.0 mm or less in the thickness direction.

According to the above configuration, since the two side surfaces (hereinafter sometimes referred to as wide side surfaces) of the enlarged portion bulge out to both sides in the thickness direction, and since the thickness (height difference) of the step structure formed on at least one of the wide side surfaces is small, it is difficult to restrict the method of holding the ultrasound probe, that is, it is easy to allow a variety of holding methods. On the other hand, since the step structure is provided, in a case in which the ultrasound probe is pressed against the living body or a case in which the ultrasound probe is removed from the living body, the examiner's fingertip can easily catch on at least one side surface. In the specification of the present application, “flat” means a state in which there is no structure that can catch on fingertips, that is, a state in which the surface is substantially smooth. For example, in a case in which a certain region includes a seam between two members, and the seam can be ignored from the viewpoint of fingertip catching, the region can be said to be a flat region.

According to experiments conducted by the present inventors, the following several points have been found. First, the portion of each wide side surface of the enlarged portion that is relatively likely to come into contact with the fingertip is the rear region of each wide side surface. Second, it is basically unnecessary to add a structure for the fingertips to catch on the portions of the rear region close to the two elongated side surfaces (the two corner portions), whereas it is necessary to add a structure for the fingertips to catch on the intermediate portion of the rear region (the portion between the two corner portions). Thirdly, in providing a structure for catching the fingertips, in a case in which the structure has a large unevenness (large height difference), the examiner is likely to feel uncomfortable in a case of employing a specific holding method. The above configuration was created based on these findings.

In an embodiment, the two side surfaces each have the above-mentioned front region and the aforementioned rear region. According to this configuration, the fingertip can easily catch on the enlarged portion regardless of the orientation of the ultrasound probe.

In an embodiment, the height of the first step, the height of the second step, and the height of the third step are all 1.0 mm or less in the thickness direction. According to this configuration, the examiner does not feel uncomfortable even in a case in which various holding methods are employed.

In an embodiment, the width of the recess portion in the central axis direction is greater than the width of the protrusion portion in the central axis direction. From the viewpoint of fingertip catching, it is better for the width of the protrusion portion to be relatively narrow. For example, in a case in which the same fingertip comes into contact with both the first step and the second step at the same time, the fingertip is more likely to be prevented from slipping. On the other hand, from the viewpoint of fingertip catching, it is better for the width of the recess portion to be relatively wide, and specifically, the width of the recess portion is determined so that the fingertip can be inserted inside the recess portion.

In an embodiment, at least one side surface includes a flat one region provided on one side of the step structure in the width direction, and a flat other region provided on the other side of the step structure in the width direction. The step structure is surrounded by a front region, a rear end region, one region, and the other region. According to this configuration, it is easy to employ various holding methods while obtaining catching in a specific local region.

In an embodiment, the protrusion portion has one end part and another end part spaced apart from each other in the major axis direction. In the protrusion portion, the height of one end part in the thickness direction gradually decreases as it approaches the one region, and the terminal end of the one end part is seamlessly continuous with the one region. In the protrusion portion, the height of the other end part in the thickness direction gradually decreases as it approaches the other region, and the terminal end of the other end part is seamlessly continuous with the other region. According to this configuration, it is easier to employ various holding methods.

In an embodiment, the recess portion has one end part and another end part spaced apart from each other in the major axis direction. In the recess portion, one end part is seamlessly continuous with one region, and the other end part is seamlessly continuous with the other region. According to this configuration, at the time of changing the holding method, it is easy to slide the fingertip inserted in the recess portion in the width direction, and the examiner does not feel uncomfortable in this case. Furthermore, in a case in which an acoustic jelly adheres to the inside of the recess portion, it becomes easier to wipe off the acoustic jelly.

In an embodiment, the length of the protrusion portion in the width direction is equal to or greater than half the maximum width of the enlarged portion in the width direction. According to this configuration, this allows for a variety of holding methods, while also making it possible to catch the fingertips on the protrusion portions (specifically, the first step and/or the second step) as necessary.

In an embodiment, the length of the third step in the width direction is smaller than the length of the protrusion portion in the width direction. Since there is a corner portion near the narrowed portion in the enlarged portion, even in a case in which the third step is shortened, the likelihood of the fingertip slippage is reduced.

In an embodiment, the transducer array has an electronic scanning direction. The transducer array is composed of a plurality of transducers arranged in the electronic scanning direction. Each of the plurality of transducers is composed of a plurality of elements arranged in the thickness direction. At least one substrate is accommodated across the enlarged portion and the narrowed portion and comprises an electronic circuit connected to the transducer array.

In a case in which the transducer array consists of a plurality of elements arranged two-dimensionally, it is necessary to provide an electronic circuit within the ultrasound probe to control the operation of the transducer array; in other words, it is necessary to secure a space within the ultrasound probe to dispose a substrate on which the electronic circuit is mounted. From the viewpoint of operability of the ultrasound probe, it is desirable to bring the center of gravity position of the ultrasound probe close to the distal end surface. Due to this background, the distal end portion is becoming enlarged. As the distal end portion becomes enlarged, it becomes more difficult to prevent fingertip slippage while allowing for a variety of holding methods; however, this can be achieved by providing a step structure in the rear region of the wide side surface (desirably the intermediate portion as a specific local region in the rear region).

### (2) Details of Embodiment

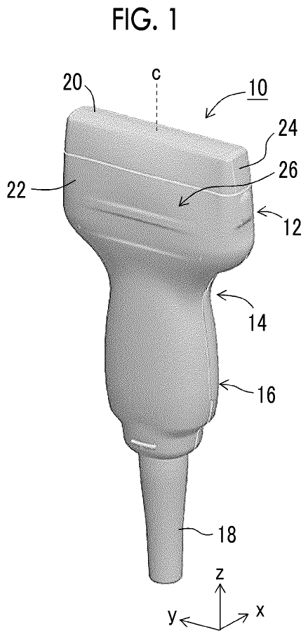

FIG. 1 shows an ultrasound probe according to an embodiment. An ultrasound probe 10 is used during ultrasound examination of a living body. The ultrasound probe 10 is connected to an ultrasound diagnostic apparatus main body (not shown). In a case in which the ultrasound probe 10 is used, the ultrasound probe 10 is held by the hand of an examiner. In FIG. 1, an x direction is the thickness direction, a y direction is the width direction, and a z direction is the central axis direction. The z direction is parallel to a central axis c of the ultrasound probe 10.

The ultrasound probe 10 has an enlarged portion 12, a narrowed portion 14, and a body portion 16. The enlarged portion 12, the narrowed portion 14, and the body portion 16 constitute a probe case, which is made of, for example, resin. The enlarged portion 12 is a distal end portion that is enlarged in the y direction and the z direction. In the narrowed portion 14, the width in the y direction is narrower than those of the enlarged portion 12 and the body portion 16, and the width in the x direction is also narrower. The body portion 16 is a portion that extends in the z direction. The narrowed portion 14 and the body portion 16 correspond to a grip portion. A cable 18 is pulled out from the body portion 16.

A transducer array is provided within the enlarged portion 12. Ultrasound is transmitted from the transducer array to the living body, and the reflected waves from the living body are received by the transducer array. The enlarged portion 12 has a distal end surface 20. The distal end surface 20 has an ultrasonic wave transmissive region corresponding to the transducer array. The distal end surface 20 intersects with respect to the z direction and faces the living body. More specifically, the central portion of the distal end surface 20 is orthogonal to the central axis c.

In the embodiment, the transducer array is a 1.25D transducer array, a 1.5D transducer array, or a 1.75D transducer array. The transducer array has a major axis direction and a minor axis direction. The major axis direction is the electronic scanning direction. In the shown configuration example, the major axis direction is parallel to the y direction, and the minor axis direction is parallel to the x direction. In the major axis direction, the aperture size is varied, ultrasound beam forming is performed, and the ultrasound beam is electronically scanned. In the minor axis direction, the aperture size may be varied, ultrasound beam forming may be performed, or the ultrasound beam may be deflected. As will be described later, for electronic control in the minor axis direction (for example, aperture switching control), a plurality of electronic circuits connected to the transducer array are provided within the ultrasound probe 10. As the transducer array, a 1D transducer array may be provided.

More specifically, the transducer array is composed of a plurality of transducers arranged in the major axis direction. Each transducer is composed of a plurality of elements (vibration elements) arranged in the minor axis direction. Although the ultrasound probe according to the embodiment is a linear ultrasound probe, the technology according to the embodiment may be applied to a convex ultrasound probe.

The enlarged portion 12 has two wide side surfaces 22 and two elongated side surfaces 24. The two wide side surfaces 22 each intersect with respect to the x direction. More specifically, each wide side surface 22, viewed as a whole, intersects with respect to the x direction at an angle close to 90 degrees. The two elongated side surfaces 24 each intersect with respect to the y direction. More specifically, each elongated side surface 24, viewed as a whole, intersects with respect to the y direction at an angle close to 90 degrees.

The two wide side surfaces 22 bulge outward on both sides in the x direction. Each wide side surface 22 extends in the y direction and the z direction and, except for a portion, has a flat form, that is, a smooth form. A portion is a step structure 26 provided on each of the wide side surfaces 22. The step structure 26 will be described in detail later.

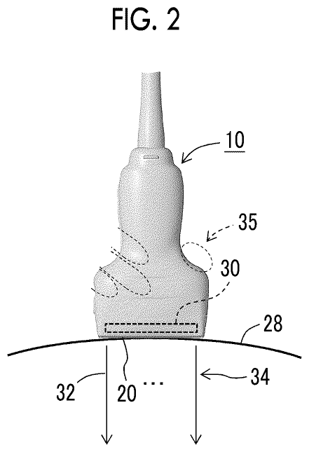

FIG. 2 shows an example of a state in which the ultrasound probe 10 is used. The ultrasound probe 10 is held by the hand 35 of an examiner. The distal end surface 20 of the ultrasound probe 10 is in contact with the surface 28 of the subject. Prior to use of the ultrasound probe 10, an acoustic jelly is applied to the distal end surface 20 and to the surface 28 of the subject. An ultrasound beam 32 is formed by the transducer array 30. A beam scanning surface 34 is formed by electronic scanning with the ultrasound beam 32. The method of holding the ultrasound probe is selected depending on the posture of the subject, the examination site, the cross section to be observed, the examiner's preference, and the like.

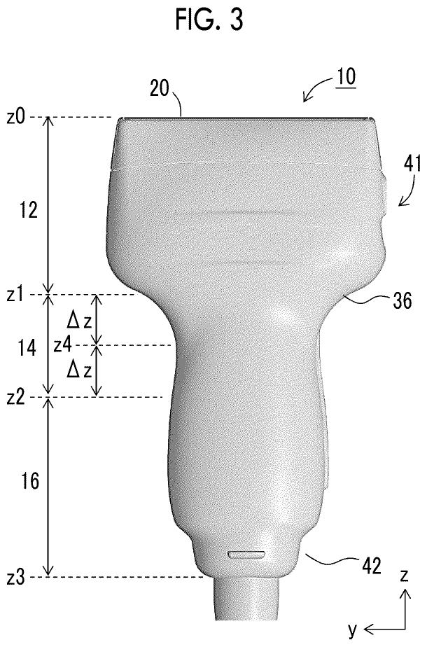

FIG. 3 shows the front of the ultrasound probe 10. In FIG. 3, z0 indicates the position of the distal end surface in the enlarged portion 12. 20 is the reference position. z1 indicates the position of the boundary between the enlarged portion 12 and the narrowed portion 14. For example, as viewed from the x direction, the position of the inflection point or midpoint of an S-shaped curved edge line 36 is z1. z4 indicates the position on the xy cross section where the circumference length is minimum. The distance between z1 and z4 is represented as Δz. The narrowed portion 14 has a width of 2×Δz in the z direction. z2 indicates the position of the boundary between the narrowed portion 14 and the body portion 16. z3 indicates the position of the terminal end of the body portion 16.

In a case in which the distance from z0 to z3 is L, the distance from z0 to z4 is within a range of 0.3L to 0.6L, for example. The distance from z0 to z1 is, for example, within a range of 30 to 50 mm, and the width of the distal end surface 20 in the y direction is, for example, within a range of 50 to 70 mm. The width of the distal end surface in the x direction is, for example, within a range of 8 to 20 mm. The minimum circumference length of the narrowed portion 14 is, for example, in the range of 80 to 100 mm. The maximum circumference length of the body portion 16 is, for example, in the range of 90 to 110 mm. Any numerical values given in the specification of the present application are merely examples. Reference numeral 41 denotes an uneven structure used in a case of attaching the puncture device. Reference numeral 42 denotes a rear end part to which a magnetic sensor is attached.

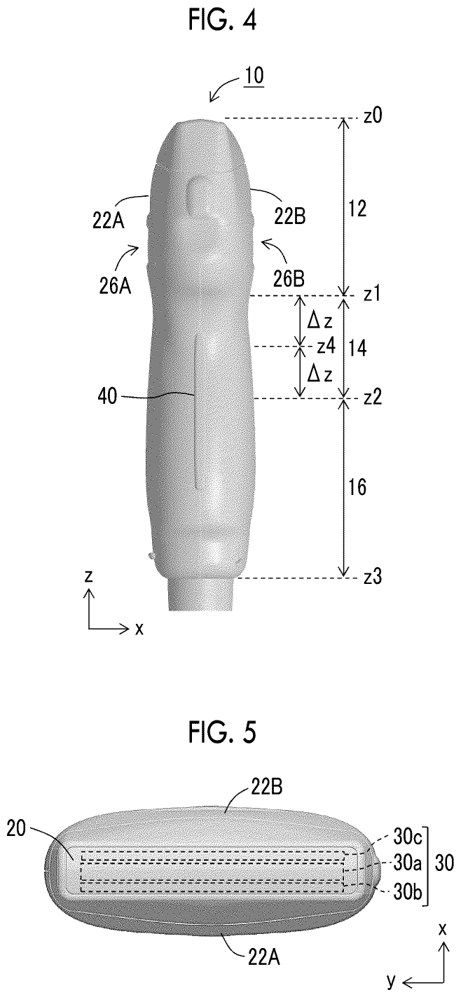

FIG. 4 shows the side surface of the ultrasound probe 10. In FIG. 4 as well, the ranges of the enlarged portion 12, the narrowed portion 14, and the body portion 16 are specified by z0 to z4. One step structure 26A is formed on one wide side surface 22A, and the other step structure 26B is formed on the other wide side surface 22B. Reference numeral 40 denotes a mark for identifying the reference end (start end) in the electronic scanning direction.

FIG. 5 shows a planar surface of the ultrasound probe. The distal end surface 20 has a rectangular form. Reference numeral 30 denotes a transducer array. The transducer array 30 is composed of a plurality of transducers arranged in the major axis direction (y direction). In the shown example, each transducer is composed of three elements arranged in the minor axis direction (x direction). From another perspective, the transducer array 30 is composed of three element rows 30a, 30b, and 30c arranged in the minor axis direction (x direction). Each of the element rows 30a, 30b, and 30c is composed of a plurality of elements arranged in the major axis direction (y direction).

In FIG. 5, one wide side surface 22A and the other wide side surface 22B bulge outward on both sides in the x direction. One wide side surface 22A and the other wide side surface 22B are each gently curved along the y direction and also gently curved along the z direction.

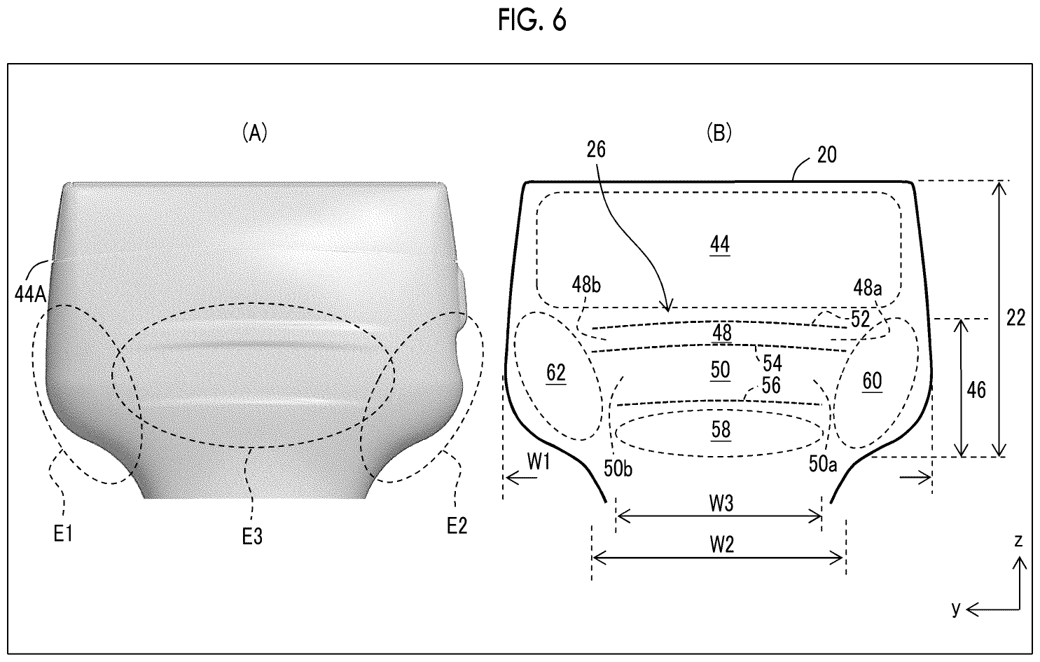

FIG. 6 shows the enlarged portion. (A) shows an image of the enlarged portion.

According to experiments by the present inventors, assuming a variety of holding methods, the regions on the wide side surface that are likely to come into contact with the examiner's fingertips are a plurality of regions E1, E2, and E3 away from the distal end surface. Of the plurality of regions E1, E2, and E3, the regions E1 and E2 each include a corner portion or are close to a corner portion, and it is basically unnecessary to add an uneven structure to the regions E1 and E2. On the other hand, in the region E3, the fingertips tend to slip, and it is necessary to add an uneven structure there.

In FIG. 6, (B) shows a schematic diagram of the wide side surface 22. The wide side surface 22 consists of a front region 44 and a rear region 46 arranged in the x direction. The front region 44 is continuous with the distal end surface 20 and is a flat region. The front region 44 includes a seam 44A between the two members, but from the viewpoint of action on the fingertips, the seam 44A can be ignored.

The rear region 46 is provided rearward of the front region 44 in the z direction. The rear region 46 includes a step structure 26, one region 60, the other region 62, and a rear end region 58. In the embodiment, the step structure 26 has a protrusion portion 48 and a recess portion 50. The protrusion portion 48 is a band-shaped region extending along the y direction. The recess portion 50 is a band-shaped region extending along the y direction.

More specifically, the step structure 26 has a first step 52, a second step 54 and a third step 56. The first step 52 is a step that corresponds to the boundary between the front region 44 and the protrusion portion 48, and is an upward step as viewed from the −z direction. The second step 54 is a step that corresponds to the boundary between the protrusion portion 48 and the recess portion 50, and is a downward step as viewed from the −z direction. The third step 56 is a step that corresponds to the boundary between the recess portion 50 and the rear end region 58, and is an upward step as viewed from the −z direction. Each of the steps 52, 54, and 56 is actually a slope.

One region 60 is a flat region provided on one side of the step structure 26 in the y direction. The other region 62 is a flat region provided on the other side of the step structure 26 in the y direction. The rear end region 58 is a flat region provided on the rear side of the step structure 26 in the z direction. The step structure 26 is surrounded by the front region 44, one region 60, the other region 62, and the rear end region 58. The width of the protrusion portion 48 in the z direction is smaller than the width of the recess portion 50 in the z direction. The width of the recess portion 50 is determined so as to allow the insertion of a fingertip.

The protrusion portion 48 has one end part 48a and the other end part 48b spaced apart in the y direction. The height of one end part 48a in the x direction gradually decreases as it approaches one region 60, and the terminal end of the one end part 48a is seamlessly continuous with the one region 60, that is, smoothly. The height of the other end part 48b in the x direction gradually decreases as it approaches the other region 62, and the terminal end of the other end part 48b is seamlessly continuous with the other region 62.

The recess portion 50 has one end part 50a and the other end part 50b spaced apart in the y direction. On the other hand, the one end part 50a is seamlessly continuous with the one region 60. The other end part 50b is seamlessly continuous with the other region 62. The rear end region 58 is continuous with the one region 60 and the other region 62. The recess portion 50 (more accurately, the bottom surface of the recess portion), the one region 60, the other region 62, and the rear end region 58 all together form a single flat region.

W1 indicates the maximum width of the enlarged portion in the y direction. W2 indicates the length of the protrusion portion 48 in the y direction (the length of the first step 52 and the length of the second step 54). W3 indicates the length of the third step 56 in the y direction. The relationship W1>W2>W3 is established. In the embodiment, furthermore, the relationship W2>W1×½ is established. W1 is, for example, in the range of 50 to 70 mm. In the rear region 46, the step structure 26 extending in the y direction is formed in the intermediate portion in the y direction. The step structure 26 is surrounded by a flat region.

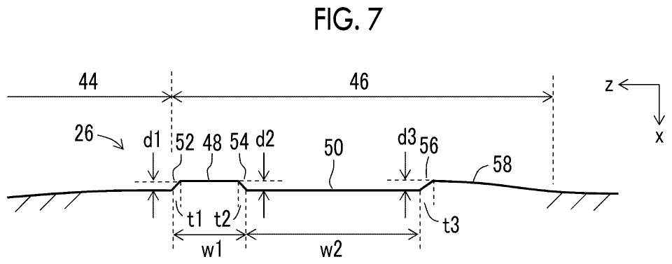

FIG. 7 shows a cross section of the step structure 26. Reference numeral 44 denotes a front region and reference numeral 46 denotes a rear region. As already described, the step structure 26 has a protrusion portion 48 and a recess portion 50, that is, a first step 52, a second step 54, and a third step 56. In FIG. 4, the first step 52, the second step 54, and the third step 56 are expressed in a slightly exaggerated manner.

The height (maximum height) of the first step in the x direction is indicated as d1. The height (maximum height) of the second step in the x direction is indicated as d2. The height (maximum height) of the third step in the x direction is indicated as d3. From the viewpoint of allowing for a variety of holding methods, that is, from the viewpoint of reducing the restrictions on holding methods caused by the step structure, in other words, from the viewpoint of maintaining the smoothness of the enlarged portion as a whole, d1, d2, and d3 are each set to 2 mm or less, and in the embodiment, are 1 mm or less. From the viewpoint of generating an appropriate catch in a specific local region, each of d1, d2, and d3 is set to, for example, 0.3 mm or more.

In FIG. 7, w1 indicates the width of the protrusion portion 48 in the z direction (more accurately, the width of the base of the protrusion portion 48), and w2 indicates the width of the recess portion 50 in the z direction (more accurately, the width of the bottom surface of the recess portion 50). In the embodiment, w1<w2. w2 is set within the range of 5 to 15 mm, for example. w1 is set within the range of 2 to 6 mm, for example.

As viewed from the −y direction, the first step 52 forms an upward slope, and the width of the upward slope in the z direction is t1. As viewed from the −y direction, the second step 54 forms a downward slope, and the width of the downward slope in the z direction is t2. As viewed from the −y direction, the third step 56 forms an upward slope, and the width of the upward slope in the z direction is t3. t1, t2, and t3 are set within the range of 0.3 to 1.0 mm, for example. The lower end and the upper end of each slope may be rounded. In that case, each width in the y direction may be defined with respect to the midpoint of the roundness.

In the ultrasound probe according to the embodiment, a step structure having a small height difference is provided in the intermediate portion of the rear region on each wide side surface, and the step structure is surrounded by a flat region. Therefore, the examiner is unlikely to feel uncomfortable even in a case in which various holding methods are employed, and it is possible to effectively prevent the fingertips from slipping on each wide side surface.

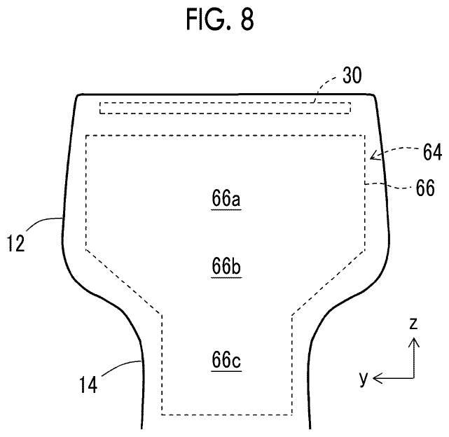

FIG. 8 shows a substrate group 64 provided in the ultrasound probe. The substrate group 64 is provided across the enlarged portion 12 and the narrowed portion 14. The substrate group 64 is composed of a plurality of substrates 66 arranged in the x direction, and these substrates 66 basically have the same form. Each substrate 66 has a plurality of electronic circuits mounted thereon. Each electronic circuit is for controlling the operation of the transducer array 30.

Each substrate 66 is composed of a front part 66a, an intermediate part 66b, and a rear part 66c. The front part 66a extends in the y direction and the z direction and has a rectangular form. The intermediate part 66b has a trapezoidal form. The rear part 66c has a rectangular form, but it is smaller than the front part. The enlarged portion 12 is enlarged in order to increase the area of each substrate 66 and to bring the substrate group 64 closer to the living body. Accordingly, the step structure appropriately prevents fingertips from slipping, which can easily occur.

In the above-mentioned step structure, two or more protrusion portions and two or more recess portions may be provided. The above-mentioned step structure may be provided on an ultrasound probe having a curved distal end surface. It is desirable to provide the same step structure on two wide side surfaces, but the above-mentioned step structure may be provided on one of the two wide side surfaces. In that case, the other wide side surface may be a flat surface, or another step structure may be provided on the other wide side surface.

Explanation Of References

What is claimed is:

1. An ultrasound probe having a central axis direction, a width direction, and a thickness direction that are orthogonal to each other, the ultrasound probe comprising:
an enlarged portion that accommodates a transducer array;
a narrowed portion that is continuous with the enlarged portion in the central axis direction; and
a body portion that is continuous with the narrowed portion in the central axis direction,
wherein the enlarged portion has a distal end surface that intersects with respect to the central axis direction and includes an ultrasonic wave transmissive region, and two side surfaces that bulge out to both sides in the thickness direction, each of which intersects with respect to the thickness direction and extends along the central axis direction and the width direction,
at least one side surface of the two side surfaces has a front region that is continuous with the distal end surface and is flat, and a rear region that is continuous with the front region in the central axis direction and has a step structure,
the step structure has
a protrusion portion that is provided on a rear side of the front region in the central axis direction, extends along the width direction, and has a band shape,
a recess portion that is provided on a rear side of the protrusion portion in the central axis direction, extends along the width direction, allows a fingertip of an operator to be inserted, and has a band shape, and
a rear end region that is provided on a rear side of the recess portion in the central axis direction and is flat, and
a height of a first step between the front region and the protrusion portion, a height of a second step between the protrusion portion and the recess portion, and a height of a third step between the recess portion and the rear end region are all 2.0 mm or less in the thickness direction.

2. The ultrasound probe according to claim 1,
wherein the two side surfaces each have the front region and the rear region.

3. The ultrasound probe according to claim 1,
wherein the height of the first step, the height of the second step, and the height of the third step are all 1.0 mm or less in the thickness direction.

4. The ultrasound probe according to claim 1,
wherein a width of the recess portion in the central axis direction is greater than a width of the protrusion portion in the central axis direction.

5. The ultrasound probe according to claim 1,
wherein the at least one side surface includes
one region that is provided on one side of the step structure in the width direction and is flat, and
another region that is provided on another side of the step structure in the width direction and is flat, and
the step structure is surrounded by the front region, the rear end region, the one region, and the other region.

6. The ultrasound probe according to claim 5,
wherein the protrusion portion has one end part and another end part spaced apart from each other in the width direction,
a height of the one end part in the thickness direction of the protrusion portion gradually decreases as the one end part approaches the one region, and a terminal end of the one end part is seamlessly continuous with the one region, and
a height of the other end part in the thickness direction of the protrusion portion gradually decreases as the other end part approaches the other region, and a terminal end of the other end part is seamlessly continuous with the other region.

7. The ultrasound probe according to claim 5,
wherein the recess portion has one end part and another end part spaced apart from each other in the width direction,
the one end part of the recess portion is seamlessly continuous with the one region, and
the other end part of the recess portion is seamlessly continuous with the other region.

8. The ultrasound probe according to claim 1,
wherein a length of the protrusion portion in the width direction is equal to or greater than half a maximum width of the enlarged portion in the width direction.

9. The ultrasound probe according to claim 1,
wherein a length of the third step in the width direction is smaller than a length of the protrusion portion in the width direction.

10. The ultrasound probe according to claim 1,
wherein the transducer array has an electronic scanning direction,
the transducer array is composed of a plurality of transducers arranged in the electronic scanning direction,
each of the plurality of transducers is composed of a plurality of elements arranged in the thickness direction, and
at least one substrate including an electronic circuit connected to the transducer array is accommodated across the enlarged portion and the narrowed portion.
